(19) United States
(12) Patent Application Publication
(10) Pub. No.: US 2021/0177261 A1
(43) Pub. Date: Jun. 17, 2021

(54) SYSTEM, METHOD, AND COMPUTER-ACCESSIBLE MEDIUM FOR MAGNETIC RESONANCE VALUE DRIVEN AUTONOMOUS SCANNER

(71) Applicant: The Trustees of Columbia University in the City of New York, New York, NY (US)

(72) Inventors: Sairam GEETHANATH, New York, NY (US); Keerthi SRAVAN RAVI, New York, NY (US); John Thomas Vaughan, JR., New York, NY (US)

(21) Appl. No.: 17/170,173

(22) Filed: Feb. 8, 2021

Related U.S. Application Data
(63) Continuation of application No. PCT/US2019/046136, filed on Aug. 12, 2019.
(60) Provisional application No. 62/717,860, filed on Aug. 12, 2018.

(51) Int. Cl. A61B 5/00; A61B 5/055; A61B 6/03; G01R 33/561; G16H 40/67; G16H 30/20; G16H 30/40; G16H 50/20; G16H 50/50; G16H 50/70; G16H 15/00; G06N 3/08; H04L 29/06

(52) U.S. Cl. CPC A61B 5/0042; H04L 63/0428; A61B 5/7267; A61B 5/0035; A61B 5/0022; A61B 5/0013; A61B 6/032; A61B 6/037; G01R 33/5615; G16H 40/67; G16H 30/20; G16H 30/40; G16H 50/20; G16H 50/50; G16H 50/70; G16H 15/00; G06N 3/08; A61B 5/055

(57) ABSTRACT

Exemplary system, method and computer-accessible medium for remotely initiating a medical imaging scan(s) of a patient(s), can include, for example, receiving, over a network, encrypted first information related to first parameters of the patient(s), determining second information related to image acquisition second parameters based on the first information, generating an imaging sequence(s) based on the second information, and initiating, remotely from the patient(s), the medical imaging scan(s) based on the imaging sequence(s). The medical imaging scan(s) can be a magnetic resonance imaging (“MRI”) sequence(s). The image acquisition second parameters can be MRI acquisition parameters, and the imaging sequence(s) can be a gradient recalled echo (“GRE”) pulse sequence(s).

### CROSS-REFERENCE TO RELATED APPLICATION(S)

This application relates to and claims priority from U.S. Patent Application No. 62/717,860, filed on Aug. 12, 2018, the entire disclosure of which is incorporated herein by reference.

### FIELD OF THE DISCLOSURE

The present disclosure relates generally to magnetic resonance (“MR”), and more specifically, to exemplary embodiments of exemplary system, method, and computer-accessible medium for providing, utilizing and/or facilitating, autonomous MR.

### BACKGROUND INFORMATION

In a typical MR image setting, qualified personnel registers a patient to be imaged and activates and runs the MR scan. However, while qualified personnel may be easily found to perform this function in certain countries, in many areas of the world, it can be difficult to find qualified personnel to operate a MR scanner.

Over the last forty-one years, Magnetic Resonance Imaging (“MRI”) has proven to be a critical component of diagnostic healthcare as an imaging modality. (See, e.g., Reference 1). Structural, functional and metabolic MRI generate insightful information for an accurate diagnosis of a wide range of pathologies. (See, e.g., Reference 2). However, the accessibility of MRI as a healthcare service ranges from being prohibitive to scarcely available depending on the geography being considered. (See, e.g., Reference 3). A suitable metric for evaluating the geographical accessibility of MRI is scanner density, measured as number of scanner units per million people (“pmp”). Globally, a scanner density is believed to be imbalanced. (See, e.g., Reference 4).

India—the second most populous country (e.g., 1.32 billion)—has a scanner density of less than 1 pmp. (See, e.g., Reference 5). Eleven (11) countries in Africa (e.g., with populations ranging from 0.735 million to 67.51 million) have no scanners. (See, e.g., Reference 41). In developing countries, lack of educational facilities and/or the high costs involved in imparting technical training has resulted in a lack of skilled manpower needed to operate MRI systems. (See, e.g, Reference 6). While imaging has been shown to increase the utilization of facility-based rural health services, and to impact management decisions (see, e.g., Reference 6), MRI requires technical expertise to setup the patient, as well as to acquire, visualize and interpret data. The availability of such local expertise in certain geographies such as sub-Saharan Africa (“SSA”) is challenging. (See, e.g., References 4 and 6). Most countries in SSA have few or no radiologists, with the majority being deployed in cities and metropolitan areas. Therefore, there is a critical, unmet need to make MRI more accessible globally while controlling the cost factors of an MR exam. (See, e.g., Reference 3).

In countries with higher scanner densities than the global average of 5.3 pmp, inefficient workflows and usage of MRI result in challenges related to financial and temporal accesses. (See, e.g., Reference 7). Overutilization of imaging services is acknowledged in the medical industry, primarily driven by financial incentives of the healthcare system, defensive medicine and patient expectations, to name a few. (See, e.g., Reference 8). For example, ‘protocol creep’ is a term that refers to the practice of performing exams by modifying protocols on a case-by-case basis because a standard catalogue of protocols does not exist. This non-uniformity of protocols increases drastically when multiple departments function under a single system. (See, e.g., Reference 8). This results in the subject undergoing exams for longer durations than initially intended, and directly translates to higher costs to be borne by the subject and/or other stakeholders.

Managing costs in addition to improving quality and outcomes is beneficial to maximizing the value of an imaging service. (See, e.g., Reference 9). This motivation is well captured by the definition of MR value—defined as the ratio of actionable diagnostic information to the costs incurred (e.g., including the time involved in acquiring that information). (See, e.g., Reference 10).

Thus, it may be beneficial to provide exemplary system, method, and computer accessible medium for MR value driven autonomous scanner which can overcome at least some of the problems and: or issues presented herein above.

### SUMMARY OF EXEMPLARY EMBODIMENTS

Exemplary system, method and computer-accessible medium for remotely initiating a medical imaging scans) of a patient(s), can include, for example, receiving, over a network, encrypted first information related to first parameters of the patient(s), determining second information related to image acquisition second parameters based on the first information, generating an imaging sequence(s) based on the second information, and initiating, remotely from the patient(s), the medical imaging scan(s) based on the imaging sequence(s). The medical imaging scan(s) can be a magnetic resonance imaging (“MRI”) sequence(s). The image acquisition second parameters can be MRI acquisition parameters, and the imaging sequence(s) can be a gradient recalled echo (“GRE”) pulse sequence(s),

In some exemplary embodiments of the present disclosure, the GRE pulse sequence(s) can be generated based on a radio frequency (“RF”) offset(s). The RF offset(s) can be generated using a convolutional neural network(s) (“CNN”). The CNN(s) can be trained based on a single axial slice of an image of a brain of a further patient(s). The MRI acquisition parameters can include (i) a flip angle, (ii) an echo time, and/or (iii) a repetition time. A Bloch equation simulation can be performed to generate simulated results of a magnetic resonance (MR) scan of the patient(s) based on the first parameters and the image acquisition parameters. The imaging sequence(s) can be generated based on the simulated results. A MR value(s) can be generated based on the simulated results. The medical imaging scan(s) can be initiated only if the MR value is above a predetermined value.

In certain exemplary embodiments of the present disclosure, the image acquisition second parameters can be determined using a lookup tablet(s). The medical imaging scan(s) can include, e.g., (i) a positron emission tomography scan (i) a computed tomography scan, and/or (ii) an x-ray scan. The first parameters can include, e.g., (i) health information for the patient(s), (ii) geographical information of the patient(s), (iii) a height of the patient(s), and/or (iv) a weight of the patient(s). A unique key can be assigned to the patient(s). An image(s) can be generated based on the medical imaging scan(s) using cloud computing. A report(s) regarding the exemplary results of the medical imaging scan(s) can be generated and provided to the patient(s)An initiation request can be received from the patient(s) and the medical imaging scan(s) can be initiated e.g., only after the initiation request can be received.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary flow diagram according to an exemplary embodiment of the present disclosure;

FIG. 2 is an exemplary flow diagram of a single sequence exam according to an exemplary embodiment of the present disclosure;

FIG. 3 is an exemplary diagram illustrating situation report coding according to an exemplary embodiment of the present disclosure;

FIG. 4A is an exemplary diagram illustrating interactions and file interfaces between the three modules of the exemplary system, method, and computer accessible medium according to an exemplary embodiment of the present disclosure;

FIG. 4B is an exemplary diagram of various exemplary scenarios that can be performed using the exemplary system, method, and computer-accessible medium according to an exemplary embodiment of the present disclosure;

FIG. 5 is an exemplary flow diagram of the exemplary system, method, and computer-accessible medium supporting more than one clinical application according to an exemplary embodiment of the present disclosure;

FIG. 6 is an exemplary flow diagram of a real-world deployment of the exemplary system, method, and computer-accessible medium according to an exemplary embodiment of the present disclosure;

FIG. 7 is an exemplary flow diagram illustrating an exemplary operation of an autonomous MRI scan according to an exemplary embodiment of the present disclosure;

FIG. 8 is a set of exemplary images illustrating intelligent slice planning according to an exemplary embodiment of the present disclosure;

FIGS. 9A-9C are exemplary images illustrating image reconstruction according to an exemplary embodiment of the present disclosure;

FIGS. 10A-10C are exemplary graphs illustrating a quantitative analysis of image reconstructions according to an exemplary embodiment of the present disclosure;

FIG. 11A is an exemplary graph illustrating the total time for the reconstructions shown in FIGS. 10A-10C according to an exemplary embodiment of the present disclosure;

FIGS. 11B, 11C and 11D are exemplary graphs illustrating exemplary acquisition times for the reconstructions shown in FIGS. 10A, 10B, and 10C, respectively, according to an exemplary embodiment of the present disclosure;

FIG. 12 is an exemplary diagram of an autonomous MRI intelligent physical system according to an exemplary embodiment of the present disclosure;

FIG. 13 is an exemplary flow diagram of a method for remotely initiating a medical imaging scan of a patient according to an exemplary embodiment of the present disclosure; and

FIG. 14 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the. illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary embodiments of the present disclosure may be further understood with reference to the following description and the related appended drawings. The exemplary embodiments of the present disclosure relate to a remotely operated imaging system. For example, as discussed herein, a magnetic resonance imaging (“MRI”) apparatus can include a configuration or a setup, which can be remotely operated and controlled. Thus, there is no need for trained personnel to be located at the imaging site. The exemplary embodiments of the present disclosure are described herein with reference to a MRI apparatus, although those having ordinary skill in the art will understand that the exemplary embodiments of the present disclosure may be implemented on any imaging apparatus including, X-ray machines, computed tomography scanners, positron emission tomography scanners, etc.

Autonomous and time-efficient acquisition, reconstruction and visualization procedures to maximize magnetic resonance (“MR”) hardware usage and exemplary solutions that reduce reliance on human operation of MR systems can alleviate some of the challenges associated with the requirement/absence of skilled human resource. (See, e.g., References 6, 11, and 12). The exemplary system/apparatus according to an exemplary embodiment of the present disclosure can include an Autonomous MRI (“AMRI”). The exemplary methods according to an exemplary embodiment of the present disclosure described herein can be used to modify existing scanners to be an Intelligent Physical System (“IPS”). (See, e.g., Reference 14). An IPS can be characterized by cognizance, taskability, ethicality, adaptability and its ability to reflect (see, e.g., Reference 14), and can perform its task with minimal or no human intervention. In one example, the entire procedure for performing an MR exam can be autonomous, and thus facilitates a check on the ‘table time’. An AMRI user is not required to possess any particular technical knowledge to perform an MRI examination. This can be different from a remote exam that can require the presence of a well-trained MR technician or radiologist at a different site, and therefore AMRI can mitigate the demand for skilled manpower.

In order to begin using the exemplary system, method and computer-accessible medium, a person can initiate the patient registration process by interacting with the software either via voice or other input modalities on a smart device, referred to as a “remote” or a “remote device.” The clinical application can also be selected. It can be important to note that the user operating the remote need not be physically far away from the MR system.

The patient registration details (e.g., protected health information) can be encrypted and transferred to the cloud. The cloud can assign a unique key to the patient. The patient's historical health information, and other contextual information (e.g., geographical information, etc.) can be utilized to define MR protocol an optimized protocol). A cloud-based Bloch equation simulator can be run to simulate the results of the proposed MR protocol.

The MR system's localizer can be executed to sample the current state, and such information can be compiled into a situation report (“Sitrep”). This Sitrep can be communicated to the cloud. Based on the time remaining, the simulation's results and the Sitrep, an MR value can be derived. The MR value can be on a scale of 1-10. The user can be presented with this MR value as the theoretical maximum that earl be achieved in current conditions and asked if they would like to proceed, if the user agrees to proceed with the MR scan, the patient's unique key, the bare-minimum information utilized to carry out a safe scan (e.g., specific absorption rate parameters) such as the patient's height and weight, and the sequence definition can be queued as a ‘Job’. The scanner can continuously ping the cloud to retrieve the latest job. The scanner console can generate the pulse sequence on the fly (e.g., in real time), and the scan can be initiated. At the end of this sequence, another Sitrep can be generated and communicated to the cloud along with the acquired data.

An exemplary MR image reconstruction processes can be computed on the cloud, leveraging virtually infinite computing resources, and the reconstructed results can be communicated with the remote. A ‘smart report’, also generated on the cloud, can be communicated to the remote. The user operating the remote can be presented with the reconstructed images and the smart report. The smart report can include information provided by analyzing the image generated using the exemplary system/apparatus. For example, the smart report can include a diagnosis, a prognosis, a treatment plan, recommendations from a physician, or any other information related to the results of the imaging scan.

The exemplary system, method, and computer-accessible medium, according to an exemplary embodiment of the present disclosure can transform a standard MRI system into an IPS. This can facilitate the MRI system to be remotely activated, interactively invoked and self-driven to optimize a MR value. Each scan can be tailored to the patient undergoing the exam based on multiple factors, integrated into the determination of MR value. An MR value can be provided to the clinician as the ratio of actionable diagnostic information to the costs incurred (e.g., acquisition time, scheduling cost, interpretation costs, technician time, etc.). A simplified interpretation of a MR value can be defined as the ratio of Contrast-to-Noise Ratio (“CNR”) to total scanner (e.g., table) time utilized to perform the exam. A higher MR value can indicate superior and/or beneficial outcomes for the stakeholders.

ATI IPS can function autonomously if it can be characterized as being cognizant, taskable, adaptive and ethical. Cognizance can further be broken down into discrete concepts such as: (i) reflection, (ii) retention, (iii) revision and (iv) reuse. A system that can analyze the results of a just-accomplished task can be deemed reflective and subsequent updating of its knowledge base with information derived from the analyses can be considered revision. Revision and reuse of the accumulated knowledge can also constitute cognizance. A cognizant system can also be aware of its own capabilities and limitations. Thus, the exemplary system, method, and computer-accessible medium, can be taskable since it can interact with the user via multiple modalities (e.g., text input, voice commands, etc.) and it can understand commands which can be vague or high-level. The exemplary system, method, and computer-accessible medium, according to an exemplary embodiment of the present disclosure can also be adaptive since it can successfully handle discrepancies encountered during its autonomous functioning without disruption. Finally, the exemplary system, method, and computer-accessible medium, can be ethical since it can consult established societal and legal guidelines for its decisions.

### Exemplary Tripartite Software Framework

The exemplary system, method, and computer-accessible medium, according to an exemplary embodiment of the present disclosure can include three sub-packages: one each for the user node, the cloud, and the scanner. The user node can be the smart device that can interact with the user and record the issued commands, inform of the progress of the scan and present the reconstructed images. The user node may or may not be physically present in close proximity to the scanner. The cloud can host the knowledge base, generate pulse sequences, evaluate the state of the MR scanner, and transmit the commands received by the user node to the scanner and computes image reconstructions. The cloud can include any system which can have significant computing power and/or an extensive amount of data storage space. The scanner can be a combination of the scanner console and the MRI scanner system.

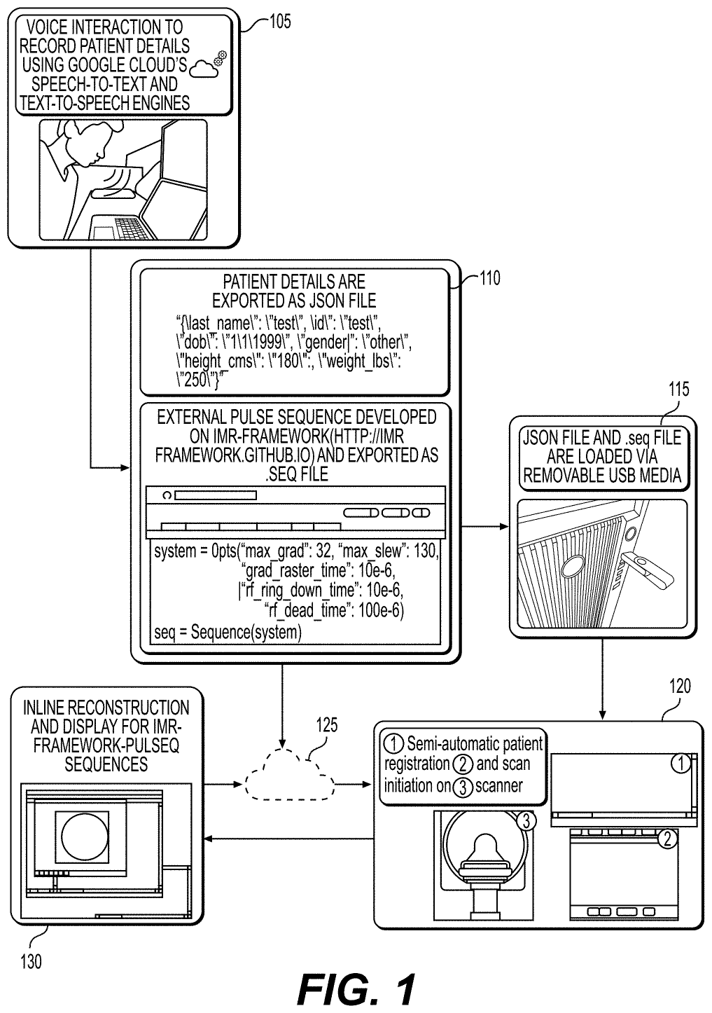

FIG. 1 shows a flow diagram according to an exemplary embodiment of the present disclosure where USB media can be used to transfer data between the user node and the scanner. For example, at procedure 105, a user (e.g., a patient) can interact with a computer system in order to record patient details. This can be performed using an exemplary speech-to-text engine and/or a text-to-speech engine. At procedure 110, patient details can be exported in an appropriate file format for the imaging procedure (e.g., a JSON file). At procedure 115, the JSON file and an imaging sequence, can be loaded using a removable storage medium (e.g., a USB flash drive). At procedure 120, a program can be activated which can perform the patient registration (e.g., using the recorded patient details), and initiate a scan on a scanner. The imaging information can be sent for inline reconstruction and display at procedure 130. As shown in FIG. 1, the patient information, and scan sequence, can be manually loaded (e.g., using a portable storage medium). Alternatively, or in addition, as shown in FIG. 1, a cloud service 125 can be utilized to share the information needed to register a patient, perform a scan, and perform a reconstruction and display of the image.

### Exemplary User Node

The exemplary user node can interact with the user via voice in a question and answer format to register patient information and other details. The user node can request the user to clarify if it encounters invalid or inappropriate commands. The user node can utilize the specification of the clinical protocol/application along with facilitated/acceptable time for the exam. This can be leveraged to optimize the exam for the MR value.

An exemplary text-to-speech (“TTS”) engine (e.g., Google's Cloud Text-to-Speech engine) can be used to convert input text into voice to prompt the user to issue commands pertaining to patient information. An exemplary Speech-to-Text (“STT”) engine can be used to convert these voice-commands issued by the user into text. The user node can record the details from the user that can be utilized to register the patient on the scanner: (i) last name, (ii) age, (iii) height and (iv) weight. A unique ID can be assigned to the patient, which can be used to successfully register a patient on the scanner. Voice interactivity with the user can characterize the system as taskable, and the ability to request the user to clarify in case of any discrepancies while registering the patient can characterize the system as adaptive.

Fernet symmetric encryption can be used to encrypt patient parameters, before being uploaded to the cloud. A new encryption key can be requested from the cloud for each exam. Since no identifiable information can be transmitted to the cloud unencrypted, the system can be characterized as ethical.

Most applications offload the compute-intensive functions to the cloud because of the capability of elastically scaling to demand offered by the organizations providing cloud services.

A neural network (e.g., a convolutional neural network, a recurrent neural network, a fully convolutional neural networks, or any other suitable neural network) can be trained to accept a single axial slice brain image as an input and produce a distance to the preferred landmark (e.g., the anterior commissure—posterior commissure line in case of the brain) as an output. The problem of computing this distance to the preferred landmark can be treated as a multiclass classification problem. This computed distance can be translated to the RF offset for the pulse sequence.

The framework can be demonstrated for a simple brain screening protocol including T₁, proton density and T₂* weighted images. The combination of acquisition parameters such as TE, TR, flip angle and number of signal averages can be chosen by referencing a lookup table (“LUT”). This combination of parameters, along with the computed RF offset can generate a Gradient Recalled Echo (“GRE”) pulse sequence on the cloud and saved as a .seq file. This sequence can then be played on the scanner. The resulting image from this acquisition can be analyzed for CNR and new parameters for the subsequent pulse sequences to be optimized based on the LUT. Pulse sequences for the subsequent Proton Density (“PD”) weighted and T₂* weighted scans can also be generated in a similar manner. The patient's last name can be masked with the unique ID that can be assigned and a scan job was issued.

The LUT can include a combination of a range of values for TE, TR, flip angle and number of slice averages. Signal intensities can be computed for all these combinations as per the spoiled GRE signal intensity equation, given by, for example:

$$S = {S}_{M} \frac{sin α ( 1 - {e}^{- \frac{TR}{T 1}} )}{( 1 - ( cos α ) {e}^{- \frac{TR}{T 1}} )} {e}^{- \frac{TE}{T 2 *}} \times \sqrt{NSA}$$

where, S_{M} can be the mean proton density of the type of human brain matter (e.g., gray, white, cerebrospinal fluid), α can be flip angle in radians, TR can be repetition time, TE can be echo time, T1 and T2* can be respective relaxation times for the human brain, and NSA can be the Number of Signal Averages. Table 1. below shows a few sample combinations of values used to create the LUT with NSA=1,

TABLE 1
Combination of ranges of values of TE, TR (e.g., in seconds) and flip
angle (e.g., in degrees) for number of slice averages set to one.

| Contrast | TE(ms) | TR (ms) | Flip angle (deg) |
|---|---|---|---|
| T₁ | 8 | 14 | 20 |
| | | 18.4 | 24.4 |
| | | — | — |
| | | — | — |
| | | 49.5 | 55.5 |
| | | 54 | 60 |
| PD | 4 | 10 | 5 |
| | | 11.1 | 6.6 |
| | | — | — |
| | | — | — |
| | | 18.8 | 18.3 |
| | | 20 | 20 |
| T₂* | 4 | 10 | 5 |
| | 4.3 | 11.1 | 6.6 |
| | — | — | — |
| | — | — | — |
| | 6.6 | 18.8 | 18.33 |
| | 7 | 20 | 20 |

Exemplary Methods/Procedures 100481 For example, all AMRI features were implemented in Python 3.6 (see, e.g., Reference 15), and related open-source libraries. The volunteer study was approved by the local institutional review board. AMRI operates in two modes: (i) standard mode—where the ‘user’ was any MR safety aware hospital worker (e.g., nurse, for example) administering the scan; self-administered mode—where the ‘user’ was any MRI safety aware subject intending to undergo the exam.

### Exemplary AMRI

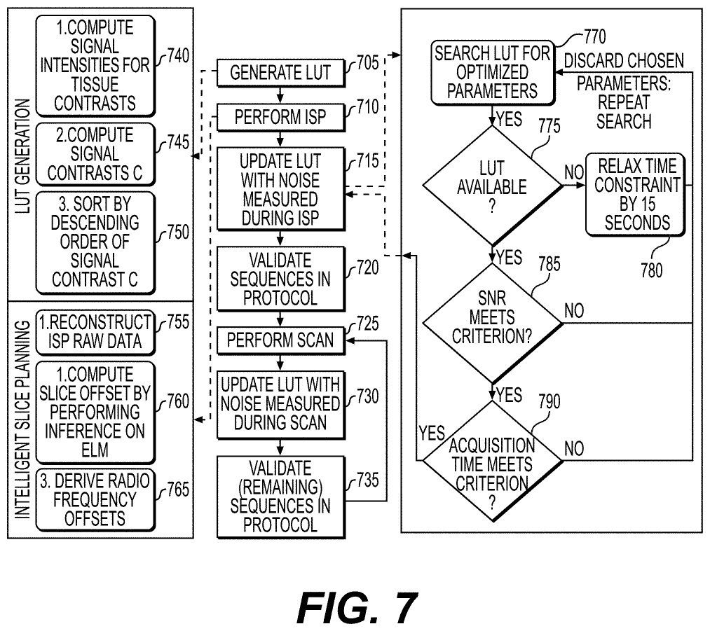

The exemplary AMRI setup/configuration consisted of three (3) components: user node, cloud and scanner. This tri-partite setup facilitated for a logical partitioning of functionalities. The user node can be any smart device that interacted with the user via one or more input modalities. Examples of such input modalities can be interacting via voice, keyboard input, a web-firm, integration with health information systems, etc. The cloud can be any system with significant compute and storage to primarily perform compute-intensive functions and host the knowledge-base. Acquisition parameters were based on those that produced best contrast while meeting SNR and acquisition time criteria to generate pulse sequences for each scan. It also communicated the user's commands to the scanner and informed the user about scan progress. The scanner was singly tasked with acquiring raw data from the subject based on the instructions from the cloud. It awaited commands from the cloud and automated the UI operation on the scanner console to initiate MR acquisitions. AMRI morphs the scanner from conventionally being a sophisticated system utilizing complex operations (e.g., slice planning, protocol edits based on SNR, contrast, image visualization, etc,) into only a data sensor. FIG. 7 illustrates the flow of operation in a typical AMRI scan.

FIG. 7 shows an exemplary flow diagram illustrating the operation of an autonomous MRI scan according to an exemplary embodiment of the present disclosure, which can include various modules for performing certain functions as described herein. For example at procedure 705, the LUT can be generated. This can be performed by computing signal intensities for tissue contrasts at procedure 740, computing signal contrasts at procedure 745, and sorting by a descending order of signal contrasts at procedure 750. At procedure 710, ISP can be performed. This can be based on reconstructing ISP raw data at procedure 755, computing a slice offset by performing inference on the ELM at procedure 760, and deriving/determining RF offsets at procedure 765. At procedure 715, the LUT can be updated with noise measured during the ISP. This can be performed by searching the LUT for optimized parameters at procedure 770. If a LUT is not available (e.g., determined at procedure 775), then time constraints can be relaxed by a certain period of time at procedure 780 (e.g., 15 seconds). If a LUT is available, then a determination can be made at procedure 785 as to whether the SNR meets certain criteria. If it does, then a determination can be made as to whether the acquisition time meets certain criteria. If it does, then this information can be passed back to the exemplary procedures to be used to validate the sequences in a protocol at procedure 720. At procedure 725, the scan can be performed. At procedure 730, the LUT can be updated with the noise measured during the scan, and at procedure 735, the remaining sequences can be validated.

### Exemplary Voice Interaction, Information Encryption and User Intervention

AMRI interacted with the user via voice in a question/answer format to register the subject. The subject's name, height (e.g., centimeters), weight (e.g., pounds), gender, age and the choice of protocol to be executed for the MR exam were recorded in this manner. The user was asked to clarify if an invalid or inappropriate response was encountered. Since AMRI's initial implementation only supported a modified brain screen protocol (see, e.g., Reference 16), the choice of protocol was inconsequential. The subject's name was masked by a 128-bit unique ID generated by Python's built-in uuid library. The subject's details were then encrypted using symmetric authenticated cryptography before being transmitted to the cloud. If AMRI could not successfully tune the protocol parameters to satisfy the SNR and time criteria, it requested the user's permission to proceed with a modified acquisition time. This subject registration was the only user I/O task of AMRI.

Google's google-cloud-python and goggle-cloud-text-to-speech libraries were leveraged to perform STT and TTS respectively. As instructed by the official documentation, a Google Cloud project was initialized and the associated API key was utilized in the STT and TTS implementations respectively. The subject information encryption was performed by leveraging the Fernet implementation provided by the cryptography library. (See, e.g., Reference 17). The URL-safe base64-encoded 32-bit secret key required for the Fernet encryption was generated by the cloud at the start of each MR exam.

### Exemplary Intelligent Slice Planning

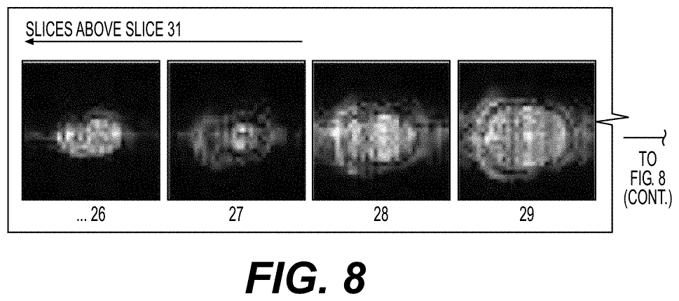

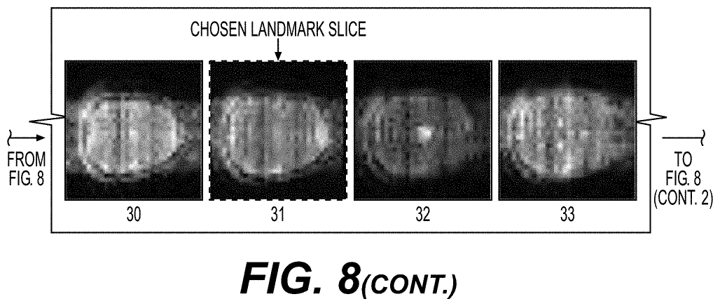

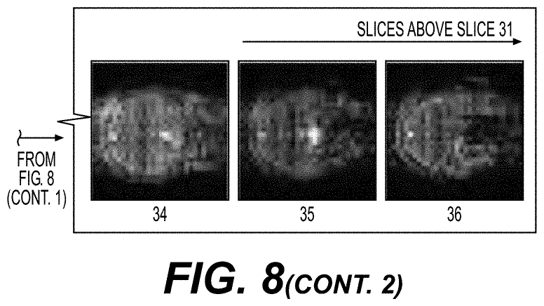

Exemplary Slice planning was treated as a multi-class classification problem and implemented using an extreme learning machine (“ELM”). (See, e.g., Reference 18). The multi-class classification problem was designed as follows (see e.g., images shown in FIG. 8): the training dataset included pairs of in-vivo axial brain images and their corresponding slice positions in a brain volume. During testing, on providing a previously unseen axial brain image the trained ELM predicted its slice position. This slice position was used to determine the distance to the chosen landmark in the brain volume, which was then utilized in slice planning as offsets to the RE pulse.

The multi-class classification implementation of ISP utilized rapid inference and prototyping, and low memory consumption. An ELM can be a single-hidden layer feedforward neural network that can be significantly faster than a traditional feedforward neural network. It can demonstrate good generalization performance because it tends to converge on the smallest training error with the smallest norm of weights. (See, e.g., Reference 19). The only tunable hyperparameter can be the number of nodes, and this can result in faster prototyping. Slice planning using an ELM was performed because of this combination of superior generalization performance, fast learning speed, low memory consumption and easy hyperparameter tuning.

The ELM included 1024 nodes activated by a sigmoid function and minimized categorial cross-entropy loss. In-vivo axial volume data of the brain was acquired using a custom localizer based on a standard GRE sequence to generate the training dataset. The acquisition parameters were: TE/TR=8 ms/15 ms, flip angle=56.7°, slice thickness=5 mm, FOV=220 mm and matrix size=32×32. Numpy (sec, e.g., Reference 20), and Scipy (see, e.g., Reference 21), libraries were used to augment the dataset to consist of 5490 samples. First, the acquired slices were rotation augmented from 30° to +30° in steps of 1° utilizing bilinear interpolator. This dataset was then replicated three times and noise derived from a uniform distribution scaled by three percent was added. Each data sample was reshaped into a row vector and thresholded to the noise computed earlier. Each row vector was zero-padded to ensure each sample in the dataset was consistently 1024 samples long. The data set was split 90%-10% for training and validation. The ELM was trained to achieve a validation accuracy of 87.5% in less than sixty seconds on a 2.5 GHz Intel Core i7, AMD Radeon R9M370X 2GB Apple MacBook Pro (Apple Inc., USA). The slice offset predicted by the ELM was multiplied by the slice-thickness to derive the RF offset. This RF offSet was utilized to design the pulse sequences for the subsequent scans.

### Exemplary Intelligent Protocolling

LUTs were constructed to accomplish intelligent pulse sequence parameter tuning and adhering to acquisition time constraints. One LUT each for the T1, T2 and T2* tissue contrasts was generated. These contained combinations of a range of repetition time, echo time and flip angle pulse sequence design parameters based on GRE and SE signal equations. (See, e.g., References 22 and 23). It also contained acquisition times, brain matter signal intensities and contrast values analytically computed for each combination of these parameters. Signal intensities of grey, white and CSF matters were computed using a spoiled-GRE signal intensity equation for each combination, and contrast values were computed as the absolute differences in signal intensities between the appropriate brain matters. Each LUT was sorted in descending order by contrast value. After each scan, a noise value was computed from the acquisition. Four 10×10 corner patches of the 32×32 image reconstruction from the ISP acquisition were averaged and multiplied by 1.25 to obtain the noise threshold value. This was performed for robustness to include any additional noise components during subsequent pulse sequences. This noise value was used to compute SNR values for each combination of parameters and appended to the LUT. For the first scan in the protocol, the noise value was computed from the ISP acquisition. A SNR of 10 dB indicates that the signal can be three times stronger than the interfering noise. However, a SNR threshold of 10 dB was not achievable with the TR, TE and flip angle acquisition parameters from the LUT. Thus, a threshold of 9 dB was chosen for the exemplary SNR criterion,

The standard exemplary AMRI exam included searching the LUTs to derive the best combination of pulse sequence design parameters for each exam that satisfied the acquisition time constraint and the SNR criterion. At the beginning of the search, the first row of each LUT containing the combination of parameters producing the best tissue contrasts was chosen. If the SNR criterion was met in all three cases, their corresponding acquisition times were summed, and the exam proceeded if the cumulative acquisition time met the time constraint. Otherwise the LUTs for T2 and T2* contrasts were alternatively traversed to derive combinations of parameters producing the next-best tissue contrasts while also meeting the SNR criterion. Subsequently, the exam proceeded only if the new cumulative acquisition time satisfied the time constraint. If the LUT was exhausted while attempting to derive a combination of parameters that is all criteria, AMRI relaxed the acquisition time constraint by 15 second increments. The LUT was then re-populated and the search was repeated after each increment. After each scan, the SNR values in the LUTs were updated with noise computed from the image reconstructions of the most-recent acquisition.

### Exemplary Open-Source File Standards

The exemplary system, method and computer-accessible medium, according to an exemplary embodiment of the present disclosure, can utilize standard and non-standard file formats. For example, a vendor-agnostic pulse sequence programming was facilitated by the Pulseq file standard (see, e.g., Reference 24), facilitating researchers and contributors to export pulse sequences designed in Matlab/Python/GPI as a ‘.seq’ file, which could be executed on three MRI vendor platform (e.g., Siemens, Bruker, GE) hardware by installing the relevant Pulseq interpreter. (See, e.g., References 25-27). Pulseq was leveraged by the cloud to generate pulse sequences based on the parameters derived from the LUT. The images reconstructed by the cloud were saved in the TIFF image format. An exemplary Sitrep file standard can be used as the medium of communication between the user node, cloud and the scanner.

### Exemplary Sitrep

The exemplary ‘Sitrep’ tile standard defines a format of communication between the user node, cloud and scanner. In military parlance, it is short for ‘situation report’—a periodic report of the current military situation, (See, e.g., Reference 28). Analogously in the AMRI setup, the Sitrep contains identifying information and a record of the sequence of events during an autonomous MR exam. Each recorded event can be a key-value pair; the key identifies the event and the value indicates the state of the event. For example, the cloud instructed the scanner to acquire data to perform ISP by issuing a command containing the value ‘True’ for the key ‘start_isp’. The Sitrep was uploaded to Google's Drive (Google Inc., USA) online file storage service, and this enabled communication between the user node and the cloud over the Internet. The cloud and scanner communicated via a copy of the Sitrep stored on the cloud.

### Exemplary Scanner

The scan procedure/job issued by the cloud can be parsed for patient information. The .seq file generated by the cloud can be copied. The PyAutoGUI Python library can simulate mouse clicks and keyboard inputs to automate graphical user interface flows. This library can be used to automate the patient registration and scan invocation flow by pattern matching against a library of screenshots that can be captured. The library of screenshots can include cropped images illustrating the patient registration flow. Once the scan is completed, the acquired data can be copied to the cloud via the OEM's (e.g., Siemens Twix) tile browsing software. The file copy flow on Twix can be automated.

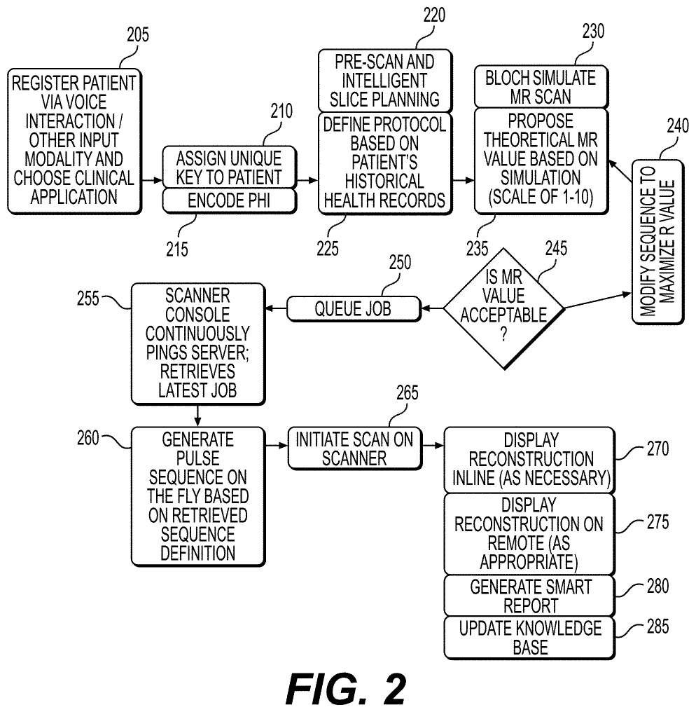

FIG. 2 shows an exemplary flow diagram of a single sequence exam according to an exemplary embodiment of the present disclosure. The user can interact with the user node via their voice to issue patient registration information. The user node can asynchronously request the cloud for an encryption key. The patient information and unique patient ID can be encrypted with the encryption key provided by the cloud. The encrypted data can be uploaded to a network or cloud-based drive. The cloud can retrieve the encrypted patient information and decrypt it. This information can be added to the database.

For example, as illustrated in FIG. 2, at procedure 205, a patient can be registered using voice input, or another input modality, and a clinical application can be selected. At procedure 210, a unique key can be assigned to the patient, and at procedure 215, protected health information (“PHI”) can be encoded. At procedure 220, a pre-scan and an intelligent slice plan can be performed. At procedure 225, a protocol can be selected or defined based on the patient information (e.g., the patient's historical health records). At procedure 230, a Bloch simulation of an MRI scan can be performed, and at procedure 235, a MR value can be selected or determined based on the simulation. At procedure 240, the MRI sequence can be modified based on the selected/determined MR value. At procedure 245, a determination can be made as to ether the MR value is acceptable. If it is, then the MRI scan can be queued at procedure 250. At procedure 255, a MR scanner console can continuously ping a server to retrieve/obtain the latest job to be performed. At procedure 260, a pulse sequence can be generated based on a retrieved sequence definition. At procedure 265, a scan can be initiated on the MRI scanner. At procedure 270, an inline reconstruction can be displayed at or near the MRI scanner, which can also be displayed remotely at procedure 275. At procedure 280, a smart report can be generated, and a knowledgebase can be updated at procedure 285.

A scan procedure/job can be issued to perform intelligent slice planning. The acquired raw data can be uploaded to the cloud, to be reconstructed. Contrast-to-noise ratio (“CNR”) can be computed based on the results of the slice planning image. RF offsets to image the target can be computed. The LUT (e.g., lookup table) can be consulted, and based on the time the user intends to spend on the MR exam, an MR value can be computed and transmitted to the user node. The user node can present the user with this MR value and ask if they still wish to proceed with the scan.

If positive (e.g., yes), a pulse sequence can be generated with the computed RF offset as one of the input parameters. A scan job can also be issued, or updated, as appropriate, by the cloud. The scanner can retrieve both the scan job and the pulse sequence and can invoke the scan. Once the scan can be completed, the acquired raw data can be uploaded to the cloud.

The cloud can retrieve the acquired raw data and reconstruct the image(s), CNR can be computed for these image(s). An updated MR value can be computed based on the CNR and the remaining time. The user node can present the user with this MR value and asks if they still wish to proceed with the scan, and also displays the reconstructed image(s), If yes, these procedures can be completed until all the scans for the exam can be completed. At the end of the exam, the cloud can generate a suggestive intelligent report. The user node can retrieve this report and present it to the user.

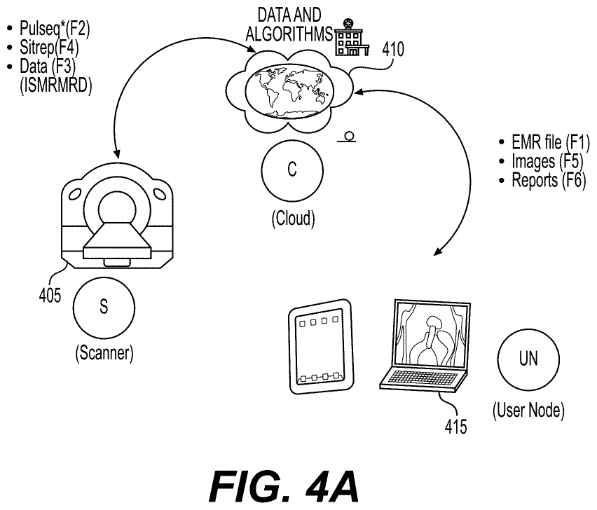

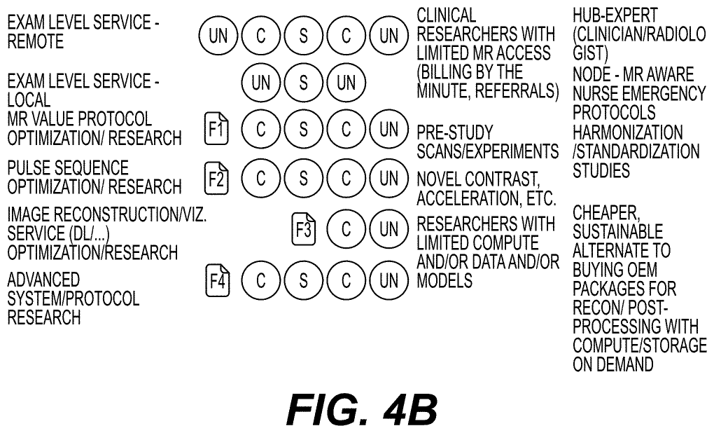

FIG. 4A shows a diagram illustrating exemplary interactions and file interfaces between the three modules of the exemplary system, method, and computer-accessible medium. For example, as shown in FIG. 4A, a scanner 405 can communicate with a user node 415 using a cloud-based service 410. FIG. 4B shows a diagram of various exemplary scenarios that can be performed using the exemplary system, method, and computer-accessible medium.

Many student researchers in the field of MRI lack easy low-cost access to MR systems for experimentation. Many hospital sites lack the utilized radiologist manpower to analyze and interpret the acquired scans, which can be directly caused by the ballooning number of medical cases utilizing MR imaging. Further, many sites lack the skilled technician manpower utilized to operate these systems. These problems could be alleviated by artificial intelligence efforts directed at enabling autonomous functioning of MR systems on a platform where MRI as an Accessible Service (“MRIAS”) could be made possible. Cloud connectivity can enable these easy access MR systems to continuously learn from vastly rich repositories of knowledge that can result in better decisions and subsequently better outcomes in real-world cases.

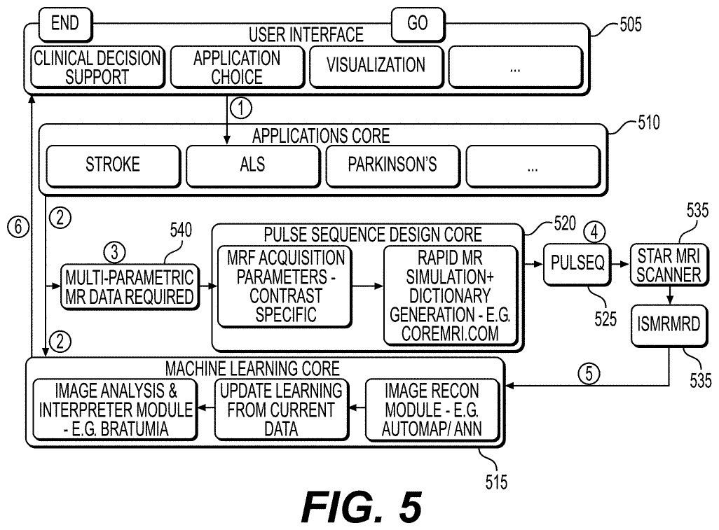

FIG. 5 shows a flow diagram of the exemplary system, method, and computer-accessible medium supporting more than one clinical application according to an exemplary embodiment of the present disclosure. For example, at procedure 505, a scan process can be initiated. At procedure 510, various application cores can be utilized based en the scan to be performed. (e.g., for a stroke, Parkinson's disease, etc.) At procedure 515, a machine learning core can be engaged. At procedure 520, a pulse Sequence design core can be utilized to facilitate the selection or determination of a pulse sequence to be used. At procedure 520, the pulse sequence can be finalized. At procedure 530, the MRI scanner can be started. At procedure 535, a data file of the scan (e.g., a ISMRMRD) can be generated. This data tile can be used to update the machine learning core.

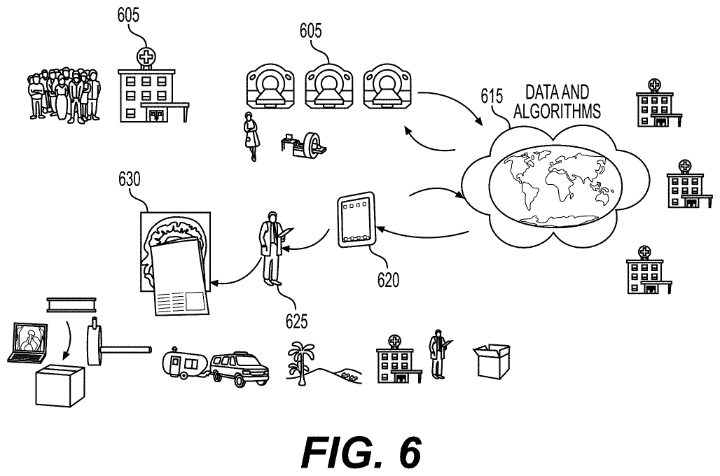

FIG. 6 shows a diagram illustrating real-world deployment of the exemplary system, method, and computer-accessible medium according to an exemplary embodiment of the present disclosure. For example, a clinician 625 can perform a comprehensive exam 630 on a patient either remotely or in-person and can view the results remotely on his/her smart device 620. The patient can be located remotely at a health facility 605, and can be imaged using a scanner 610. The information from the scanner can be stored and/or processed in a cloud environment 615.

### Exemplary Interfacing File Standards

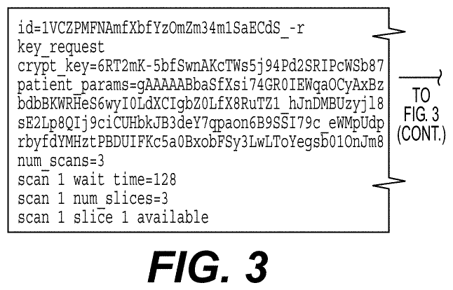

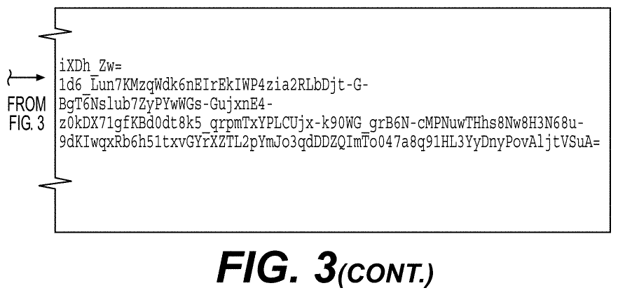

For example, a is number of (e.g., six) exemplary file standard can be used. This can include the established Pulseq file standard for pulse sequences, the proposed Sitrep file standard for autonomous MR status communication and operation, the existing Electronic Medical Record (“EMR”) for subject specific information/reporting and ISMRMRD raw data file standards. The images can be delivered in the DICOM format. These standards can facilitate universal creation, sharing, storing of these data types as well as interacting with the autonomous scanner framework, or the intelligent MR framework responsible for the operation of the autonomous scanner, demonstrated here. The Sitrep standard can define a file format in which the current state of the MR system can be saved. Sitrep can be used to define standards for the flow of control data between the user node, the cloud and the scanner. Control data can be defined as the requests or commands issued by the user node, the cloud or the scanner, indicating to proceed with the next step in the operational flow. For instance, a user node can request the cloud to generate an encryption key via the ‘key_request’ statement, and the cloud can instruct the scanner to initiate the MR scan via the ‘start scan’ command. FIG. 3 shows a portion of an exemplary Sitrep file.

### Exemplary in Vivo Experiments

In the exemplary experiment, the user node was an Apple Mac-Book Pro, the cloud was an Apple iMac Pro, and the scanner was a Siemens Prisma 3T (Siemens Healthineers, USA). The cloud and scanner were connected via a local area network.

Three experiments were designed and performed on four subjects to demonstrate AMU's cognizance, taskability, adaptability, ethicality and the capacity to reflect. The experiments differed in the imposed acquisition time constraint (e.g., denoted as minutes: seconds). After every experiment, the patient table position was reset. The first experiment 22:30) demonstrated a scenario in which AMRI can utilize acquisition parameters producing the best contrast while meeting the SNR constraint (e.g., corresponding to the ‘best’ choice of parameters). The second experiment (e.g., 13:30) demonstrated a scenario wherein AMRI could not choose the ‘best’ choice of parameters as the time constraint would not be satisfied. Therefore, the LUT was consulted to derive a combination of parameters that met the SNR criterion while also satisfying the time constraint. The acquisition times after AMRI performed the two experiments totaled to 22:4 and 13:26 respectively. In the third experiment (11:30), the LUT was exhausted in attempting to derive a combination of parameters that met both the SNR criterion and the time constraint. Therefore, AMRI relaxed the time constraint in steps of 15 seconds until it derived a choice of parameters meeting the SNR criterion. The resulting acquisition time was 12:00, and the user's consent was requested to proceed with the modified time constraint. The acquisition time was 11:56 and the exam completed in 18:45. This experiment was designed to demonstrate the characteristic of being cognizant: the system was aware of not meeting the prescribed requirements and attempted to derive a set of working parameters by relaxing a certain condition.

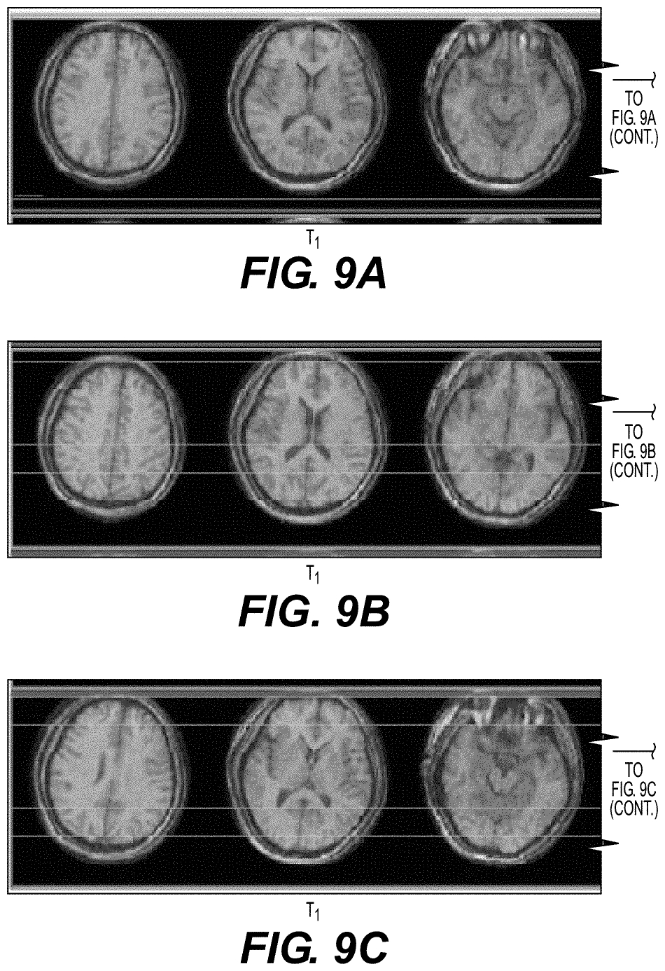

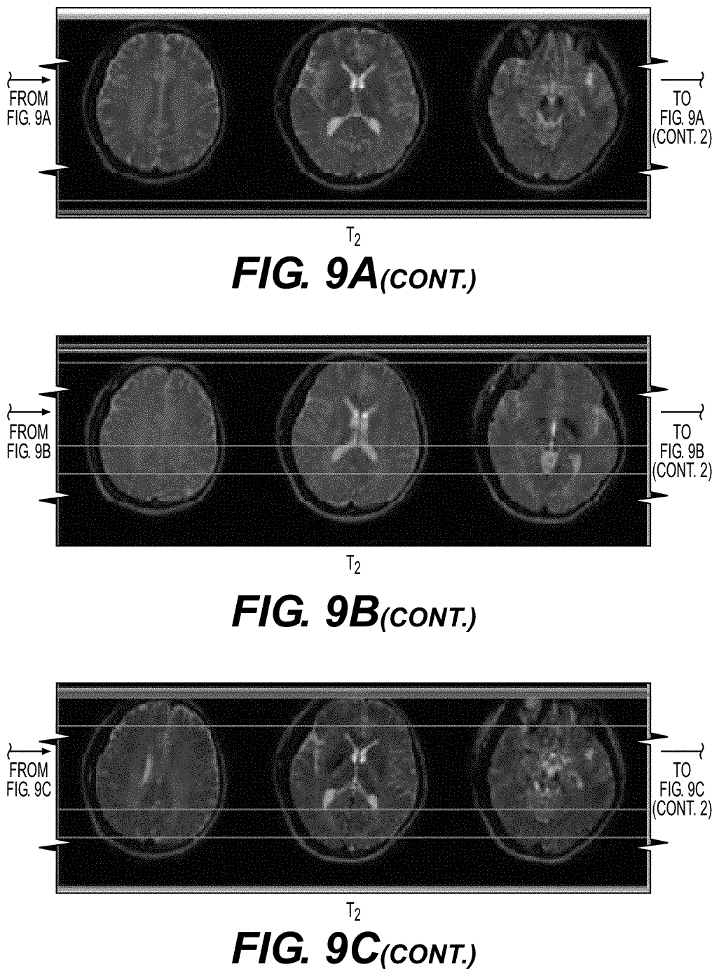

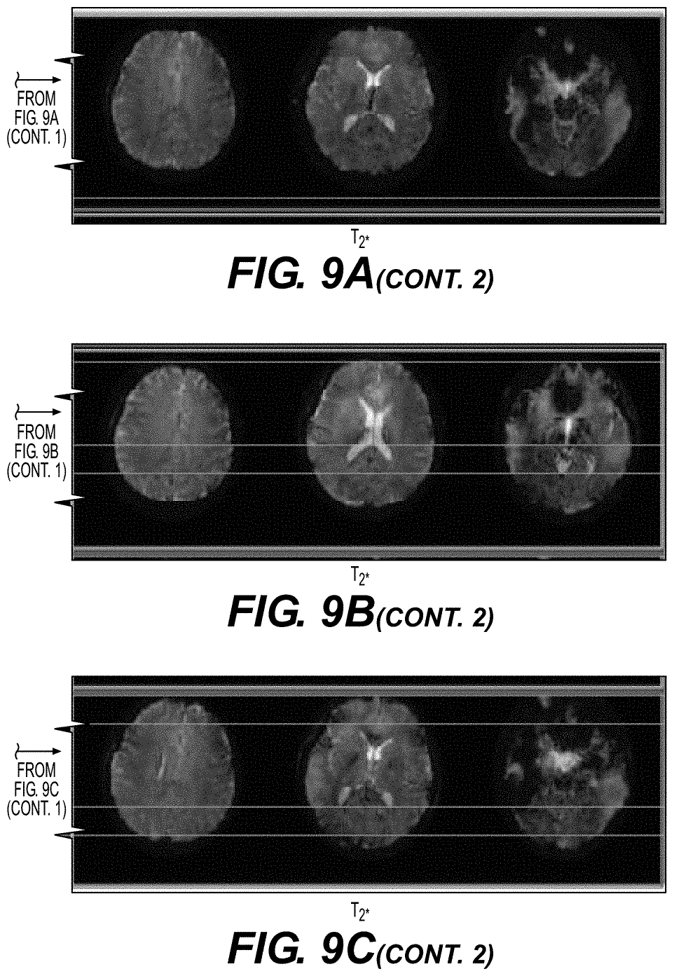

FIGS. 9A-9C illustrate exemplary images that show the activation instants of the user node cloud and scanner across the three experiments. Experiment 1 is shown in the images of FIG. 9A, experiment 2 is shown in the images of FIG. 9B, and experiment 3 is shown in the images of FIG. 9C. The first instance of activation can be when the user node requests the cloud tier an encryption key once the user begins registering the subject at the user node. The back and forth communication between the three AMRI components are marked (e.g., as illustrated in the images shown in FIG. 8), and the experiments end with the user viewing the reconstructed images on the user node. The table time for the performance of the two experiments were 28:16 and 19:51 respectively. Table time was defined as the time spent by the subject in the scanner, inclusive of the communication overheads between the user node, cloud and scanner and the time spent registering the subject. FIGS. 9A-9C further show the image reconstructions of a representative data set across the three experiments show T1, T2 and T2* contrasts. The position of the patient table was reset at the end of each experiment. It can be observed that the slices in each of the three experiments can be similar, achieved through ISP.

### Exemplary Data Analysis

SNR analysis was performed by taking the ratio of mean signal intensity from the brain to noise averaged on four 2 pixel×2 pixel corner patches on the image reconstructions. Tissue matter contrast analysis was performed by manually drawing region-of-interest (“ROI”) masks to compute absolute differences in signal intensities between white and grey matter (T₁ and T₂) and CSF and grey matter (T₂*). The T₁, T₂ and T₂* contrast images were referenced to draw binary masks for white matter, grey matter and CST respectively.

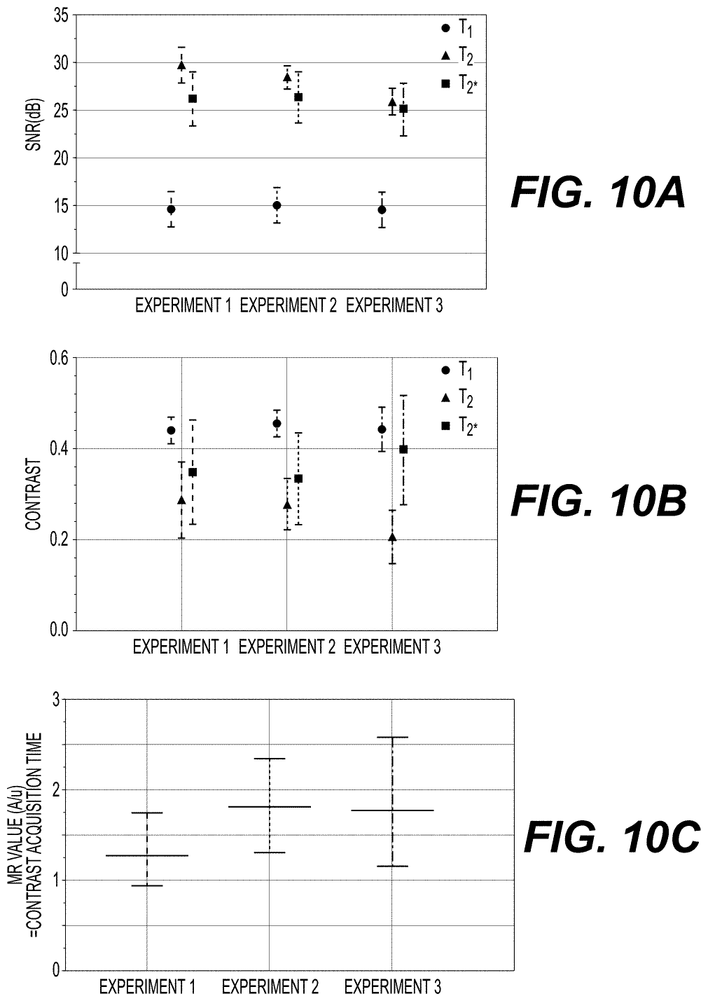

FIGS. 10A-10C are quantitative analysis plots of SNR, image contrast and MR value. MR value can be defined as the ratio of actionable diagnostic information to time spent acquiring said information. A simplified definition of MR value was optimized: the ratio of contrast achieved to the acquisition time. FIG. 10A shows an exemplary graph which indicates that SNR values were consistent within a standard deviation of 3dB. FIG. 10B illustrates an exemplary graph which indicates that contrast values for each experiment were consistent within a standard deviation of 0.12. FIGS. 10C shows an exemplary graph of achieved MR values and theoretical range of MR values. Theoretical maximum and minimum were computed as the ratios of maximum contrast to smallest acquisition time and minimum contrast to largest acquisition time.

### Exemplary Self-Administered Exam

With inclusion of inputs from audio-visual (“A/V”) accessories, the exemplary AMRI facilitated the user to perform a self-administered brain screen exam. The set up for the self-administered exam utilized a Siemens thirty-two-channel DirectConnect head coil and an MR-safe plastic chair. First, the user voice-interacted with AMRI to record registration details. Subsequently, the user landmarked the head coil and then climbed onto the patient table with the aid of the plastic chair as a stepping stool. The user then issued a voice command via MR-safe communication peripherals (e.g., OptoAcoustics FOMRI-III+microphone, OptoAcoustics, Israel) to begin the MR exam. The user was intimated of the progress of the scan via an MR-sale display placed behind the scanner, which could be read via a mirror fixed to the head-neck coil. The DirectConnect head coil was setup with the A/V accessories prior to the exam and did not require further manual operation during the exam. At the end of the exam, the patient table position was moved out to facilitate the user to exit the scanner. An illustration of the self-administered MR setup can be found online.

### Exemplary Scanner as an Online Service

An online form can be utilizes to upload a ‘.seq’ file generated using pypulseq (see, e.g., Reference 26), where an available phantom can be chosen, an available receive coil can be chosen, and a request to run a scan can be submitted. AMRI can be used to obtain the uploaded tile, perform the scan and share the raw data with the user at the listed email address. Various exemplary online storage services can be used to receive the uploaded ‘.seq’ tiles and host the reconstructed images.

### Exemplary Discussion

The LUTs were sorted in descending order of contrast values. The TE, TR and flip angle acquisition parameters were chosen so as to satisfy the SNR and time criteria. In this way, AMRI optimized each MR exam for MR value.

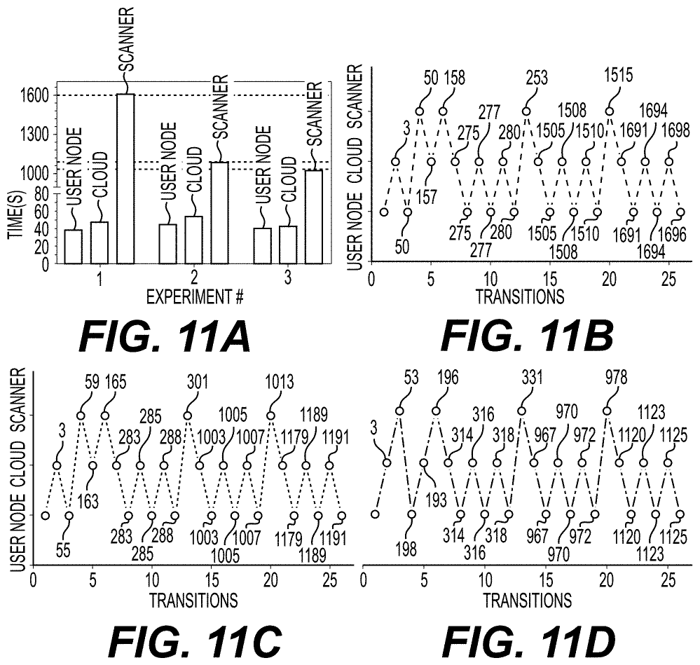

FIG. 11A shows an exemplary graph of the cumulative time spent by each AMRI component during the course of an autonomous MR exam. FIGS. 11A-11D show exemplary activity timing diagrams for experiments 1-3. For example, each node indicates a particular step in the AMRI exam, and the number of seconds spent is indicated next to each node. It can be observed that the most amount of time is spent by the scanner during the data acquisition step. All experiments incur an average communication overhead of 30.12% of the total performance time. This can be attributed to the delays incurred in automating the GUI and the length of the file-check intervals when receiving acquired raw data from the scanner. The times indicated in FIGS. 11A-11D are inclusive of communication overheads.

### Exemplary AMRI as an Intelligent Physical System

An IPS is characterized by cognizance, taskability, ethicality, adaptability and its ability to reflect. (See, e.g., Reference 14). In accordance with the above definition, a cognizant MR scanner can be aware of its capabilities and limitations in performing exams and protocols. A taskable MR scanner can interact with the user via one or more input modalities (e.g., voice/teat/gestures etc.) and interpret possibly high-level and vague instructions. The exemplary AMRI was designed to be cognizant of conforming to Signal to Noise Ratio (“SNR”) and time constraints. As shown in Experiment 3, AMRI was aware of not being able to meet the SNR criterion within the imposed acquisition time constraint. Therefore, it relaxed the time constraint in steps of 15 seconds until it could satisfy the SNR and acquisition time criteria. The exemplary system subsequently demonstrated taskability by requesting the user's approval to proceed with the working parameters. Also, AMRI registers subject information via voice interaction with the user and translates that information to influence its subsequent actions related to acquisition. An MR scanner can be ethical if it complies with prevailing societal and legal rules and frameworks. For patient privacy, AMRI masks the subject's name with a unique ID and encrypts the subject's registration information before uploading it to the cloud. It also leverages a Health insurance Portability and Accountability Act (“HIPAA”) compliant speech-to-text library to perform the voice interaction. The pulse sequence design tool leveraged in this work implements downstream Specific Absorption Ratio and Peripheral Nerve Stimulation checks to assure patient safety. An adaptable MR scanner can handle discrepancies encountered. AMRI requests the user to clarify misinterpreted voice commands. It can also report to the user in case of demands (e.g., with respect to acquisition time) that cannot be met. An IPS MR scanner can also have the ability to reflect and learn from past experiences—own or otherwise. The exemplary AMRI tuned pulse sequence parameters for each scan by accounting for the noise measured in the localizer or the previous scan. It also performs intelligent Slice Planning (“ISP”) based on the localizer acquisition to image a predetermined location and volume of interest.

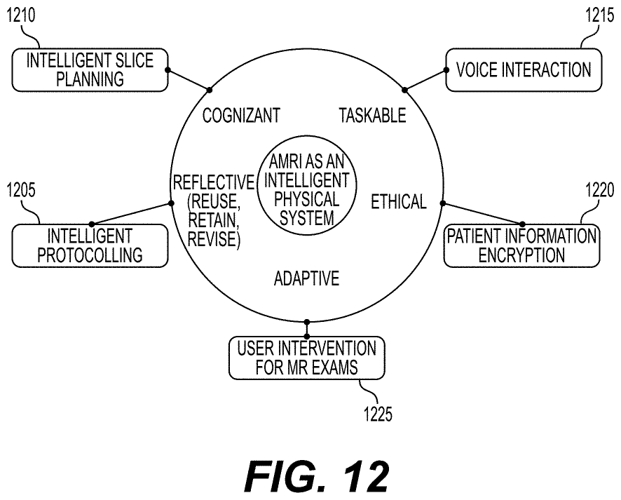

FIG. 12 maps these exemplary characteristics of an IPS to the features of AMRI. For example, FIG. 12 illustrates an exemplary diagram providing an exemplary use of intelligent protocolling 1205, intelligent slice planning 1210, voice interaction 1215, patient information encryption 1220 and user intervention for MR exams 1225.

### Exemplary MRI as an Accessible Service

Table 2 below illustrates the scenarios made possible by deploying AMRI, and demonstrates the ‘Remote’ and ‘MR acquisition’ scenarios. The user invoked scans and also viewed reconstructed images on the user node in the ‘Remote’ scenario. The user node and scanner can be in geographically distant locations and communicate via the cloud. Facilitated by Sitrep, the user can be updated of the progress of the exam throughout the procedure. The ‘MR acquisition’ scenario facilitates users without access to MR hardware to upload a ‘.seq’ file generated using pypulseq to an online form to request acquisition of raw data. The acquired raw data can be reconstructed on the cloud or shared as can be with the user.

TABLE 2
Scenarios facilitated by the software implementing the exemplary method
according to the exemplary embodiment of the present disclosure

| | | | RESEARCH |
|---|---|---|---|
| | | | ONLY/RESEARCH |
| | | | & CLINICAL |
| | SCENARIO | [symbol] | INTERESTS |
| R&D | MR | 1 [symbol] | Scanner as an |
| | ACQUISITION | \textcircled{C} Ⓢ | Online Service |
| | | \textcircled{C} \textcircled{UN} | (SOS) |
| | MR | 2 [symbol] | Deep learning |
| | RECON- | \textcircled{C} \textcircled{UN} | driven image |
| | STRUCTION | | reconstruction and |
| | | | analysis |
| | MR SYSTEMS | 3 [symbol] \textcircled{C} Ⓢ | Advanced system/ |
| | | \textcircled{C} \textcircled{UN} | protocol |
| | | | research/Cheaper, |
| | | | sustainable alternative |
| | | | for image |
| | | | reconstruction/ |
| | | | post-processing |
| | OPTIMIZING | 3 [symbol] | MR value research |
| | MR VALUE | \textcircled{C} Ⓢ | and optimization of |
| | | \textcircled{C} \textcircled{UN} | protocols/ |
| | | | Harmonization |
| | | | studies |
| | REMOTE | \textcircled{UN} \textcircled{C} | Emergency protocols |
| | | Ⓢ \textcircled{C} | |
| | | \textcircled{UN} | |
| | LOCAL | \textcircled{UN} Ⓢ | Access to clinical |
| | | \textcircled{UN} | researchers with |
| | | | limited MR access/ |
| | | | Remote exams |

The exemplary scenarios provided in Table 2 are differentiated by the files and components involved and correspond to different use cases. 1, 2 and 3 are pulse sequence exported as a ‘.seq’ file, raw data in ISMRMRD/DICOM 3.0 format, and Sitrep respectively. For example, the ‘MR Acquisition’ scenario demonstrated in this work allows users with limited access to MR hardware to acquire raw data utilizing a ‘.seq’ tile. The MR acquisition and remote scenarios have been demonstrated in this work have been implemented. UN, C, and S are abbreviations for user node, cloud and scanner respectively.

The ‘MR systems’ and ‘Optimizing MR value’ scenarios present situations that facilitate users to rapidly prototype. The method development, scan invocation and image reconstruction can be performed on a local cloud (e.g., a system with significant compute power and storage installed locally). If such a local cloud is unavailable, a standard system can be used instead. The ‘Local’ scenario is an example of such a situation. For example, 1.5 the compute-dependent tasks can be constrained by the processing power of the available system.

### Exemplary Results

A three-sequence MRI brain screening exam was remotely initiated by the user via the user node. The subject information was encrypted and uploaded to the cloud, where a unique key was assigned to the patient. This information was saved to the database. The LUT was consulted to generate the best optimized sequence for each contrast, factoring in the time that the user intended to spend on the exam. The scan was performed and the acquired raw data was uploaded to the cloud. The images were reconstructed on the cloud and were presented to the user at the user node.

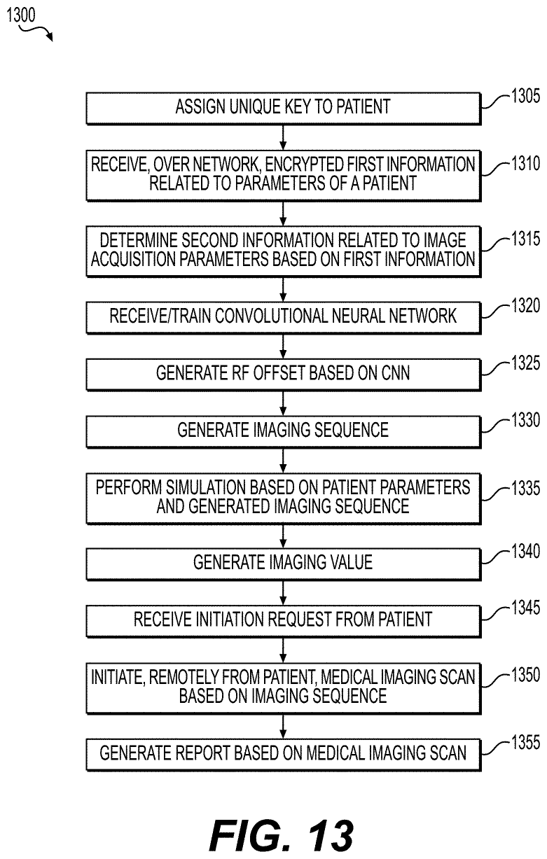

FIG. 13 shows an exemplary flow diagram of a method 1300 for remotely initiating a medical imaging scan of a patient according to an exemplary embodiment of the present disclosure. For example, at procedure 1305, a unique key can be assigned to a patient. At procedure 1310, encrypted first information related to parameters of the patient can be received over a network. At procedure 1315, second information related to image acquisition parameters can be determined based on the first information. At procedure 1320, information regarding a convolutional neural network can be received and used, which can be based on a previous training of a convolutional neural network. At procedure 1325, one or more RF offsets can be generated based on the convolutional neural network (e.g., if the imaging scan is a MRI imaging sequence). At procedure 1330, an imaging sequence (e.g., a test imaging sequence) can be generated. At procedure 1335, a simulation can be performed. based on the imaging sequence that is generated. At procedure 1340, an imaging value can be generated, which can be used to verify the simulation and determine whether or not to proceed with the scan. For example, if the determined value is too low, then the scan parameters or imaging sequence can be adjusted until a MR value is reached or exceeded. At procedure 1345, an initiation request can be received from the patient. At procedure 1350, the medical imaging scan can be initiated remotely from the patient based on the imaging sequence. At procedure 1355, a report can be generated based on the medical imaging scan.

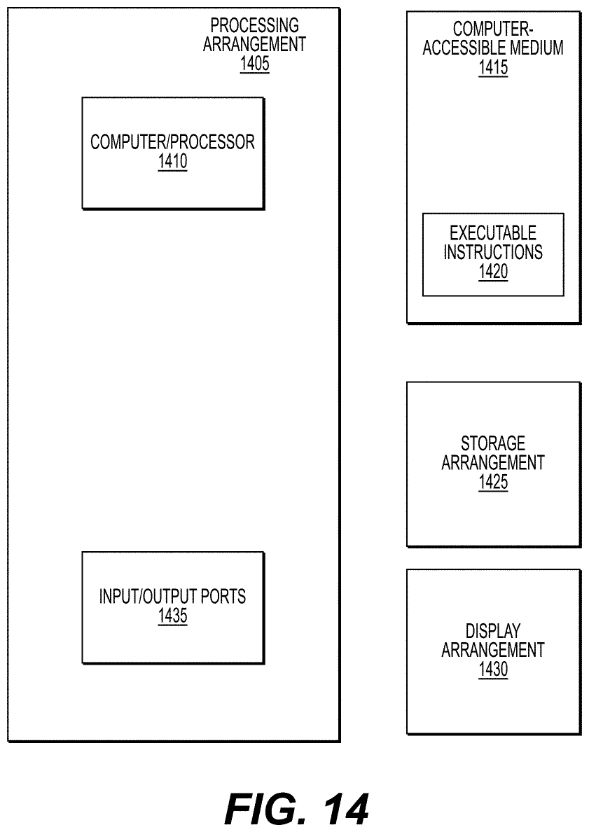

FIG. 14 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1405. Such processing/computing arrangement 1405 can be, for example entirely or a part of, or include, but not limited to, a computer/processor 1410 that can include, for example one or more microprocessors, and use instructions stored on a computer-accessible medium (e.g., RAM, ROM, hard drive, or other storage device).

As shown in FIG. 14, for example a computer-accessible medium 1415 (e.g., as described herein above, a storage device such as a hard disk, floppy disk, memory stick, CD-ROM, RAM, ROM, etc., or a collection thereof) can be provided (e.g, in communication with the processing arrangement 1405). The computer-accessible medium 1415 can contain executable instructions 1420 thereon. In addition or alternatively, a storage arrangement 1425 can be provided separately from the computer-accessible medium 1415, which can provide the instructions to the processing arrangement 1405 so as to configure the processing arrangement to execute certain exemplary procedures, processes, and methods, as described herein above, for example.

Further, the exemplary processing arrangement 1405 can be provided with or include an input/output ports 1435, which can include, for example a wired network, a wireless network, the internet, an intranet, a data collection probe, a sensor, etc. As shown in FIG. 14, the exemplary processing arrangement 1405 can be in communication with an exemplary display arrangement 1430, which, according to certain exemplary embodiments of the present disclosure, can be a touch-screen configured for inputting information to the processing arrangement in addition to outputting information from the processing arrangement, for example. Further, the exemplary display arrangement 1430 and/or a storage arrangement 1425 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.
[1] E. J. R. van Beek et al., “Value of MRI in medicine: More than just another test?,” J. Magn. Reson. Imaging, vol. 0, no. 0, pp. 1-12, 2018.
[2] R. Rao, V. Ramesh, and S. Geethanath, “Role of MRI in medical diagnostics,” Resonance, vol. 20, no. 11, pp. 1003-1011, 2015.
[3] S. Geethanath and J. T. Vaughan, “Accessible magnetic resonance imaging: A review,” Journal of Magnetic Resonance Imaging, 2019.
[4] W. H. Organization, “Global Atlas of medical devices,” 2017.
[5] Technopak, “Oncology: A Reality Check,” Technopak Healthcare Outlook, 2011.
[6] A. Burton, “Training non-physicians as neurosurgeons in sub-Saharan Africa,” Lancet Neurol., vol. 16, no. 9, pp. 684-685, 2017.
[7] M. Kawooya, “Training for Rural Radiology and imaging in Sub-Saharan Africa: Addressing the Mismatch Between Services and Population,” J. Clin. Imaging Sci., 2012.
[8] R. Kelley, “Where can $700 billion in waste be cut annually from the u.s. healthcare system?,” Thompson Reuters White Pap., 2009.
[9] P. B. Sachs, K. Hunt, F. Mansoubi, and J. Borgstede, “CT and MR Protocol Standardization Across a Large Health System: Providing a Consistent Radiologist, Patient, and Referring Provider Experience,” J. Digit. Imaging, 2017.
[10] G. D. Rubin, “Costing in Radiology and Health Care: Rationale, Relativity, Rudiments, and Realities,” Radiology, 2017.
[11] J. G. Pipe, “High-Value MRI.” Journal of Magnetic Resonance Imaging, 2018.
[12] Wang, J. C. Ye, K. Mueller, and J. A. Fessler, “Image Reconstruction is a New Frontier of Machine Learning,” IEEE Transactions on Medical Imaging. 2018.
[13] K. S. Ravi, S. Geethanath, and J. T. Vaughan Jr., “Autonomous scanning using imr-framewofk to improve MR accessibility,” in ISMRM Workshop on Accessible MRI for the world, 2019.
[14] R. Simmons, J. Donlon, and J. Yang, “Smart and Autonomous Systems (S&AS),” 2018. [Online]. Available: https://www.nsf.govsfund-ing/pgm_summ,jsp?pims_id=505325.
[15] G. van (C. voor W. en 1. (CWI)) Rossum, “Python tutorial,” Python 1995.
[16] I. (Chesterfield R. H. Bickle and B. (Royal M. H. Di Mario. “Brain screen protocol (MRI),” 2015, [Online]. Available: https://radiopaedia.org/articles/brain-screen-protocolmri-1?lang=us.
[17] P. C. Authority, “cryptography.” 2012.
[18] G.-B. Huang el al., “Extreme learning machine: Theory and applications,” Neurocomputting, 2006.
[19] P. L. Bartlett, “The sample complexity of pattern classification with neural networks: The size of the weights is more important than the size of the network,” IEEE Trans. Inf. Theory, 1998.
[20] T. E. Oliphant, A guide to NunPy. 2006.
[21] F. Jones, T. Oliphant, P. Peterson, and others, “SciPy: Open source scientific tools for Python,” Computing in Science and Engineering. 2007.
[22] M. A. Bernstein, K. F. King, and X. J. Thou, Handbook of MRI Pulse Sequences. 2004.
[23] G. D. Gillan, W. R. Nitz, and M. Brant-Zawadzki, “MP RAGE: a three-dimensional, T1-weighted, gradient-echo sequence-initial experience in the brain.,” Radiology, 1991.
[24] K. J. Layton et al., “Pulseq: A rapid and hardware-independent pulse sequence prototyping framework,” Magn. Reson. Med., 2017.
[25] N. R. Zwart and J. G. Pipe, “Graphical programming inter-face: A development environment for MRI methods,” Magn. Resort, Med., 2015.
[26] K. S. Ravi et al., “Pulseq-Graphical Programming Interface: Open source visual environment for prototyping pulse sequences and integrated magnetic resonance imaging algorithm development,” Magn. Reson. Imaging, 2018,
[27] J. F. Nielsen and. D. C. Noll, “TOPPE: A framework for rapid prototyping of MR pulse sequences,” Magn. Reson. Med., 2018.
[28] I. Quinstreet, “COMMANDER'S SITUATION REPORT [SITREP],” Army Study Guide. [Online]. Available: https://www.armystudyguide.com/content/the_tank/army_report_and_message_formats/com-manders-situation-repo.shtml.

1. A non-transitory computer-accessible medium having stored thereon computer-executable instructions for remotely initiating at least one medical imaging scan of at least one patient, wherein, when a hardware computing arrangement executes the instructions, the hardware computing arrangement is configured to perform procedures comprising:
receiving, over a network, encrypted first information related to first parameters of the at least one patient;
determining second information related to image acquisition second parameters based on the first information;
generating at least one imaging sequence based on the second information; and
initiating, remotely from the at least one patient, the at least one medical imaging scan based on the at least one imaging sequence.

2. The computer-accessible medium of claim 1, wherein the at least one medical imaging scan is at least one magnetic resonance imaging (MRI) sequence.

3. The computer-accessible medium of claim 2, wherein the image acquisition second parameters are MRI acquisition parameters, and wherein the at least one imaging sequence is at least one gradient recalled echo (GRE) pulse sequence.

4. The computer-accessible medium of claim 3, wherein the hardware computing arrangement is further configured to generate the at least one GRE pulse sequence based on at least one radio frequency (RF) offset.

5. The computer-accessible medium of claim 4, wherein the hardware computing arrangement is further configured to generate the at least one RF offset using at least one convolutional neural network (CNN).

6. The computer-accessible medium of claim 5, wherein the hardware computing arrangement is further configured to train the at least one CNN based on a single axial slice of an image of a brain of at least one further patient.

7. The computer-accessible medium of claim 3, wherein the MRI acquisition parameters include at least one of (i) a flip angle, (ii) an echo time, or (iii) a repetition time.

8. The computer-accessible medium of claim 2, wherein the hardware computing arrangement is further configured to perform a Bloch equation simulation to generate simulated results of a magnetic resonance (MR) scan of the at least one patient based on the first parameters and the image acquisition second parameters.

9. The computer-accessible medium of claim 8, wherein the hardware computing arrangement is configured to generate the at least one imaging sequence based on the simulated results.

10. The computer-accessible medium of claim 9, wherein the hardware computing arrangement is further configured to generate at least one MR value based on the simulated results.

11. The computer-accessible medium of claim 10, wherein the hardware computing arrangement is configured to initiate the at least one medical imaging scan only if the at least one MR value is above a predetermined value.

12. The computer-accessible medium of claim 1, wherein the hardware computing arrangement is configured to determine the first parameters using at least one lookup table.

13. The computer-accessible medium of claim 1, wherein the at least one medical imaging scan includes (i) a positron emission tomography scan, (ii) a computed tomography scan, or (iii) an x-ray scan.

14. The computer-accessible medium of claim 1, wherein the first parameters include (i) health information for the at least one patient, (ii) geographical information of the at least one patient, (iii) a height of the at least one patient, and (iv) a weight of the at least one patient.

15. The computer-accessible medium of claim 1, wherein the hardware computing arrangement is further configured to assign a unique key to the at least one patient.

16. The computer-accessible medium of claim 1, wherein the hardware computing arrangement is further configured to generate at least one image based on the at least one medical imaging scan using cloud computing.

17. The computer-accessible medium of claim 1, wherein the hardware computing arrangement is further configured to generate at least one report regarding the results of the at least one medical imaging scan, and provide the report to the at least one patient.

18. The computer-accessible medium of claim 1, wherein the hardware computing arrangement is further configured to:
receive an initiation request from the at least one patient, and
initiate the at least one medical imaging scan only after the initiation request is received.

19. A method for remotely initiating at least one magnetic resonance imaging (MRI) sequence of at least one patient, comprising:
receiving, over a network, encrypted first information related to parameters of the at least one patient;
determining second information related to image acquisition parameters based on the first information;
generating at least one imaging sequence based on the second information; and
using a computer hardware arrangement, initiating, remotely from the at least one patient, the at least one medical imaging scan based on the at least one imaging sequence.

20-36. (canceled)

37. A system for remotely initiating at least one magnetic resonance imaging (MRI) sequence of at least one patient, comprising:
a computing hardware arrangement configured to:
receive, over a network, encrypted first information related to parameters of the at least one patient;
determine second information related to image acquisition parameters based on the first information;
generate at least one imaging sequence based on the second information; and
initiate, remotely from the at least one patient, the at least one medical imaging scan based on the at least one imaging sequence.

38-54. (canceled)
